(19) United States
(12) Patent Application Publication
(10) Pub. No.: US 2010/0080843 A1
(43) Pub. Date: Apr. 1, 2010

(54) Natural and synthetic sulfur and selenium analogs and polymer conjugated forms thereof for the modulation of angiogenesis

(75) Inventors: Shaker Mousa, Wynantskill, NY (US); Eric Block, Niskayuna, NY (US)

Correspondence Address: HESLIN ROTHENBERG FARLEY & MESITI PC, 5 COLUMBIA CIRCLE, ALBANY, NY, 12203, US

(73) Assignee: The Research Foundation of the State University Of New York at Albany, Albany, NY (US)

(21) Appl. No.: 11/428,194

(22) Filed: Jun. 30, 2006

Related U.S. Application Data
(60) Provisional application No. 60/695,500, filed on Jun. 30, 2005.

(51) Int. Cl. A61K 31/38; A61K 31/381; A61K 31/10; A61K 31/095; A61K 9/127; A61P 35/00

(52) U.S. Cl. 424/450; 514/430; 514/446; 514/709; 514/706

(57) ABSTRACT

The present invention relates to inhibiting angiogenesis with sulfur- or selenium-containing compounds, and polymeric forms thereof, in mammals including animals and humans. Sulfur- or selenium-containing compounds, and polymeric forms thereof, can be used alone or in combination with standard therapies to inhibit angiogenesis-mediated disorders. The present invention also relates to the combined use of sulfur- or selenium-containing compounds, and polymeric forms thereof, with other anti-angiogenesis agents, with various anti-inflammatory and cytotoxic agents as well as with radio-therapeutic agents in cancer, and with laser, photodynamic therapy for ocular-related disorders such as diabetic retinopathy or age-related macular degeneration.

### CROSS REFERENCE TO RELATED APPLICATIONS

This application claims the priority of U.S. provisional application Ser. No. 60/695,500 filed Jun. 30, 2005, the contents of which are hereby incorporated by reference.

### FIELD OF THE

The present invention relates to sulfur- and selenium-containing hydrocarbon compounds, and polymeric forms thereof, and in particular, their use in modulating angiogenesis. The compounds of the invention may be used either alone or in combination with other existing anti-inflammatory, anti-angiogenesis, anti-cancer, and ocular therapies for the prevention and treatment of angiogenesis-mediated conditions.

### BACKGROUND OF THE INVENTION

Angiogenesis is the development of new blood vessels from preexisting blood vessels (Mousa, Angiogenesis Inhibitors and Stimulators: Potential Therapeutic Implications, Mousa, Landes Bioscience Inc., Georgetown, Tex.; Chapter 1; 2000. Physiologically, angiogenesis ensures proper development of mature organisms, prepares the womb for egg implantation, and plays a key role in wound healing. On the other hand, angiogenesis supports the pathological conditions associated with a number of disease states such as cancer, inflammation, and ocular diseases.

Angiogenesis or “neovascularization” is a multi-step process controlled by the balance of pro- and anti-angiogenic factors. The latter stages of this process involve proliferation and the organization of endothelial cells (EC) into tube-like structures. Growth factors such as FGF2 and VEGF are thought to be key players in promoting endothelial cell growth and differentiation. The endothelial cell is the pivotal component of the angiogenic process and responds to many cytokines through its cell surface receptors and intracellular signaling mechanisms. Endothelial cells in culture are capable of forming tube-like structures that possess lumen.

It has been proposed that inhibition of angiogenesis would be a useful therapy for restricting tumor growth. Inhibition of angiogenesis can be achieved by inhibiting endothelial cell response to angiogenic stimuli, as suggested by Folkman et al., Cancer Biology 3:89-96 (1992), where examples of endothelial cell response inhibitors such as angiostatic steroids, fungal derived products, such as fumagillin, platelet factor 4, thrombospondin, alpha-interferon, and vitamin D analogs, are described. For additional proposed inhibitors of angiogenesis, see Blood et al., Biochem. Biophys. Acta 1032:89-118; 1990, Moses et al., Science 248:1408-1410; 1990, and U.S. Pat. Nos. 5,092,885, 5,112,946, 5,192,744, and 5,202,352.

Control of angiogenesis is a complex process involving local release of vascular growth factors (Carmeliet, Ann NY Acad Sci 902:249-260; 2000), extracellular matrix, adhesion molecules, and metabolic factors (Tomanek et al., Anat Rec 261:126-135; 2000). Mechanical forces within blood vessels may also play a role (Hudlicka, Molec Cell Biochem 147:57-68; 1995). The principal classes of endogenous growth factors implicated in new blood vessel growth are the fibroblast growth factor (FGF) family and vascular endothelial growth factor (VEGF) (Pages, Ann NY Acad Sci 902:187-200; 2000). The mitogen-activated protein kinase (MAPK; ERK1/2) signal transduction cascade is involved both in VEGF gene expression and in control of proliferation of vascular endothelial cells.

The availability of a chick chorioallantoic membrane (CAM) assay for angiogenesis has provided a model in which to quantitate angiogenesis (Auerbach et al., Dev Biol. 41:391-394 (1974), Powel et al., J. Cellular Biochemistry 80:104-114 (2000); Dupont et al., Clin Exp Metastasis 19:145-153 (2002); Kim et al., Am J Pathol 156:1345-1362′(2000); Colman et al., Blood 95(2):543-550 (2000); Colman et al., J Thrombosis Haemostasis 1(1):164-173 (2003); Ali; et al., Cancer Research 60:7094-7098 (2000); Van Waes et al. Int. J Oncology 16:1189-1195 (2000); Luna et al., Lab Investigation 75:563-573 (1996). We have utilized a number of standard angiogenesis models to evaluate angiogenesis modulation by sulfur- and selenium-containing compounds, and their polymeric forms.

### SUMMARY OF THE INVENTION

The invention is based, in part, on the observation that certain sulfur-k and selenium-containing molecules or their polymeric forms are potent inhibitors of angiogenesis and inflammatory-mediated processes. These compounds inhibit the pro-angiogenic effect of pro-angiogenic factors and therefore, can be used to treat a variety of disorders characterized by excessive or inappropriate angiogenesis either alone or in combination with other existing anti-inflammatory, anti-angiogenesis, anti-cancer, and ocular therapies for the prevention and treatment of angiogenesis-mediated disorders, cancer, inflammatory, and ocular diseases.

Accordingly, in one aspect, the present invention relates to a method for inhibiting-angiogenesis comprising contacting endothelial cells with a compound of Formula I

R₁-X-R₂

wherein

### X is SO, SO₂, SeO or SeO₂;

R₁ and R₂ are substituted or unsubstituted alkyl, cycloalkyl, oxaalkyl, oxoalkyl, carboxy, alkenyl, thiaalkenyl, dithiaalkenyl, substituted or unsubstituted aryl, and wherein R₁ and R₂ are the same or different or R₁ and R₂ together form a 4-6-membered heterocyclic ring structure that may be substituted or unsubstituted.

In one aspect of the invention, the compound is a sulfone of Formula II:

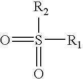

wherein
R₁ and R₂ are substituted or unsubstituted alkyl, cycloalkyl, oxaalkyl, oxoalkyl, carboxy, alkenyl, thiaalkenyl, dithiaalkenyl, substituted or unsubstituted aryl, and wherein R₁ and R₂ are the same or different or R₁ and R₂ together form a 4-6-membered heterocyclic ring structure that may be substituted or unsubstituted.

In another aspect, the compound is a sulfoxide of Formula III:

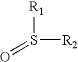

wherein
R₁ and R₂ are substituted or unsubstituted alkyl, cycloalkyl, oxaalkyl, oxoalkyl, carboxy, alkenyl, thiaalkenyl, dithiaalkenyl, substituted or unsubstituted aryl, and wherein R₁ and R₂ are the same or different or R₁ and R₂ together form a 4-6-membered heterocyclic ring structure that may be substituted, or unsubstituted.

In yet another aspect, the compound is a selenone of formula IV;

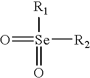

wherein
R₁ and R₂ are substituted or unsubstituted alkyl, cycloalkyl, oxaalkyl, oxoalkyl, carboxy, alkenyl, thiaalkenyl, dithiaalkenyl, substituted or unsubstituted aryl, and wherein R₁ and R₂ are the same or different or R₁ and R₂ together form a 4-6-membered heterocyclic ring structure that may be substituted or unsubstituted.

In yet another aspect, the compound is a selenoxide of formula V:

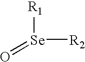

wherein
R₁ and R₂ are substituted or unsubstituted alkyl, cycloalkyl, oxaalkyl, oxoalkyl, carboxy, alkenyl, thiaalkenyl, dithiaalkenyl, substituted or unsubstituted aryl, and wherein R₁ and R₂ are the same or different or R₁ and R₂ together form a 4-6-membered heterocyclic ring structure that may be substituted, or unsubstituted.

Compounds useful for practicing the method of the invention for the inhibition of angiogenesis include but, are not limited to thiete S,S-dioxide; thiolane S,S-dioxide; di-n-butyl sulfone; 2,5-dihydrothiophene S,S-dioxide; di-n-butyl sulfone; 2,5-dihydrothiophene S,S-dioxide; 2,4-dithiapentane 2,2-dioxide; methyl trichloromethyl sulfone; 2,4-dithiapentane 2,2,4,4-tetraoxide; 2,6-dithiaspiro[3:3]heptane 2,2,6,6-tetraoxide; ajoene; diacetyl sulfone; dimethyl sulfone; diethyl sulfone; diphenyl sulfone; methylethyl sulfone; methylphenyl sulfone; ethylphenyl sulfone; diacetyl sulfoxide; dimethyl sulfoxide; diethyl sulfoxide; diphenyl sulfoxide; methylethyl sulfoxide; methylphenyl sulfoxide; ethylphenyl sulfoxide; diacetyl selenone; dimethyl selenone; diethyl selenone; diphenyl selenone; methylethyl selenone; methylphenyl selenone; ethylphenyl selenone; diacetyl selenoxide; dimethyl selenoxide diethyl selenoxide; diphenyl selenoxide; methylethyl selenoxide; methylphenyl selenoxide; ethylphenyl selenoxide; 3-(4-(methylsulfonyl)phenyl)propanoic acid; 3-(4-(methylsulfinyl)phenyl)propanoic acid; 3-(4-(methylselenonyl)phenyl)propanoic acid; 3-(4-(methylseleninyl)phenyl)propanoic acid; (2,5-bis(selenocyanatomethyl)phenyl)methanol; 2-selenophenecarboxylic acid Se; Se-dioxide; 2-selenophenecarbinol Se, Se-dioxide; p-carboxyphenyl-diphenyl selenonium and tris(p-hydroxyphenyl)selenonium salts.

In one aspect the invention relates to methods for treating a condition associated, with angiogenesis by administering to a subject in need thereof a composition comprising a sulfur and/or selenium containing compound or polymeric form or analog thereof, in an amount effective for inhibiting angiogenesis.

In a related aspect, the invention relates to sulfur- and selenium-containing compounds that are conjugate with polyvinyl alcohol, acrylic acid ethylene co-polymer, poly-lactic acid, or polyethylene glycol. Conjugation to the various polymers can be achieved via either covalent or non-covalent bonds depending on the polymer used.

The sulfur- and selenium-containing compounds and polymeric forms of the present invention are administered by parenteral, oral, rectal, or topical means, or combinations thereof. Parenteral modes of administration include, for example, subcutaneous, intraperitoneal, intramuscular, or intravenous modes, such as by catheter. Topical modes of administration can include, for example, a band-aid.

In another embodiment, the sulfur- and selenium-containing compounds of the invention can be encapsulated or incorporated into a microparticle, liposome, or polymer. The polymer can include, for example, polyglycolide, polylactide, or co-polymers thereof. The liposome or microparticle has a size of 10-1000 nanometers, and can be administered via one or more parenteral routes, or another mode of administration. In another embodiment the liposome or microparticle can be lodged in capillary beds surrounding ischemic tissue, or applied to the inside of a blood vessel via a catheter.

The sulfur- and selenium-containing compounds and polymeric forms thereof, according to the invention can also be co-administered with one or more biologically active substances that can include, for example, growth factors, vasodilators, anti-coagulants, anti-virals, anti-bacterial, anti-inflammatory; immuno-suppressants, analgesics, vascularizing agents, or cell adhesion molecules, or combinations thereof. In one embodiment, the sulfur- and selenium-containing organic molecules and polymeric forms thereof are given as a bolus injection before or after-administering one or more biologically active substance.

In a further aspect, the invention provides methods for treating a condition amenable to treatment by inhibiting angiogenesis by administering to a subject in need thereof a sulfur- and selenium-containing compounds and polymeric forms thereof as an anti-angiogenic agent (effective in inhibiting angiogenesis) and capable of blocking angiogenesis-mediated, disorders. Examples of conditions amenable to treatment by inhibiting angiogenesis include, but are not limited to, primary or metastatic tumors, diabetic retinopathy, and related conditions.

The details of one or more embodiments of the invention have been set forth in the accompanying description below. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural references unless the context clearly dictates otherwise.

### BRIEF DESCRIPTION OF THE DRAWINGS

FIG. 1 shows photomicrographs demonstrating the effect of (Z)-ajoene and (E)-ajoene on FGF2-induced angiogenesis in chick chorioalloantoic membranes.

FIG. 2 shows photomicrographs demonstrating the effect of (Z)-ajoene and (E)-ajoene on VEGF-induced angiogenesis in chick chorioalloantoic membranes.

FIG. 3 shows photomicrographs demonstrating the effect of diacetyl sulfoxide (compound 1) and diacetyl sulfone (compound 2) on FGF-induced angiogenesis in chick chorioalloantoic membranes.

### DETAILED DESCRIPTION OF THE INVENTION

All issued patents, published applications, publications and other references listed herein are hereby incorporated by reference in their entirety into the present disclosure.

Unless otherwise defined, all technical and scientific terms used herein have the same meaning as commonly understood by one of ordinary skill in, the art to which this invention pertains.

“Alkyl” refers to C1-C10 substituted, branched, unsubstituted and linear hydrocarbons potentially substituted at any of the C1-C10 positions. Examples of alkyl groups include but are not limited to methyl, ethyl, n-propyl, i-propyl, n-butyl, i-butyl- and t-butyl, pentyl, hexyl, octyl and the like. “Lower alkyl” refers to a shorter chain alkyl group, generally having eight or fewer carbon atoms.

“Cycloalkyl” refers to C3-C10 substituted or unsubstituted cyclic hydrocarbons potentially substituted at any of the C3-C10 positions. “Cycloalkyl” includes groups involving cyclic hydrocarbon functionality as a substitution of an alkyl group. Examples of cycloalkyl groups include but are not limited to c-propyl, c-butyl, c-pentyl, c-hexyl, and the like.

The term oxaalkyl refers to alkyl residues in which one or more carbons (and their associated hydrogens) have been replaced by oxygen. Examples include methoxypropoxy, 3,6,9-trioxadecyl and the like. The term, oxaalkyl, is intended as it is understood in the art [see Naming and Indexing of Chemical Substances for Chemical Abstracts, published by the American Chemical Society, ¶196, but without the restriction of ¶127(a)], i.e. it refers to compounds in which the oxygen is bonded via a single bond to its adjacent atoms (forming ether bonds); it does not refer to doubly bonded oxygen, as would be found in carbonyl groups. Similarly, thiaalkenyl refers to alkenyl residues in which one or more carbons has been replaced by sulfur; dithiaalkenyl refers to alkenyl residues in which two contiguous carbons have been replaced with sulfur.

As used herein, the terms “anti-angiogenesis agent” and “anti angiogenic agent” refer to any compound or substance that inhibits or discourages angiogenesis, whether alone or in combination with another substance.

The terms “regeneration of blood vessels,” “angiogenesis,” “revascularization,” and “increased collateral circulation” (or words to that effect) are considered as synonymous. The term “pharmaceutically acceptable” when referring to a natural or synthetic substance means that the substance has an acceptable toxic effect in view of its much greater beneficial effect, while the related the term, “physiologically acceptable,” means the substance has relatively low toxicity. The term, “co-administered” means two or more drugs are given to a patient at approximately the same time or in close sequence so that their effects run approximately concurrently or substantially overlap. This term includes sequential as well as simultaneous drug administration.

“Pharmaceutically acceptable salts” refers to pharmaceutically acceptable salts of sulfur and selenium containing organic molecules and polymeric forms thereof, which salts are derived from a variety of organic and inorganic counter ions well known in the art and include, by way of example only, sodium, potassium, calcium, magnesium, ammonium, tetra-alkyl ammonium, and the like; and when the molecule contains a basic functionality, salts of organic or inorganic, acids, such as hydrochloride, hydro bromide, tart rate, mesylate; acetate, maleate, oxalate and the like can be used as the pharmaceutically acceptable salt.

“Subject” includes living organisms such as humans, monkeys, cows, sheep, horses, pigs, cattle, goats, dogs, cats; mice, rats, cultured cells, and transgenic species thereof. In a preferred embodiment, the subject is a human. Administration of the compositions of the present invention to a subject to be treated can be carried out using known procedures, at dosages and for periods of time effective to treat the condition in the subject. An effective amount of the therapeutic compound necessary to achieve a therapeutic effect may vary according to factors such as the age, sex, and weight of the subject, and the ability of the therapeutic compound to treat the foreign agents in the subject. Dosage regimens can be adjusted to provide the optimum therapeutic response. For example, several divided doses may be administered daily or the dose may be proportionally reduced, as indicated by the exigencies of therapeutic situation.

“Administering” includes routes of administration which allow the compositions of the invention to perform their intended function, e.g., promoting angiogenesis. A variety of routes of administration are possible including, but not necessarily limited to parenteral (e.g., intravenous, intra-arterial, intramuscular, subcutaneous injection), oral (e.g., dietary), topical, nasal, rectal, or via slow releasing micro-carriers depending on the disease or condition to be treated. Oral, parenteral and intravenous administration is preferred modes of administration. Formulation of the compound to be administered will vary according to the route of administration selected (e.g., solution, emulsion, gels, aerosols, capsule). An appropriate composition comprising the compound to be administered can be prepared in a physiologically acceptable vehicle or carrier and optional adjuvant and preservatives. For solutions or emulsions, suitable carriers include, for example, aqueous or alcoholic/aqueous solutions, emulsions or suspensions, including saline and buffered media, sterile water, creams, ointments, lotions, oils, pastes and solid carriers. Parenteral vehicles can include sodium chloride solution, Ringer's dextrose, dextrose and sodium chloride, lactated Ringer's, or fixed oils. Intravenous vehicles can include various additives, preservatives, or fluid, nutrient or electrolyte replenishers (See generally, Remington's Pharmaceutical Science, 16th Edition, Mack, Ed. (1980)).

“Effective amount” includes those amounts of anti-angiogenic compounds which allow it to perform its intended function, e.g., inhibiting angiogenesis in angiogenesis-related disorders as described herein. The effective amount will depend upon a number of factors, including biological activity, age, body weight, sex, general health, severity of the condition to be treated, as well as appropriate pharmacokinetic properties. For example, dosages of the active substance may be from about 0.01 mg/kg/day to about 500 mg/kg/day, advantageously from about 0.1 mg/kg/day to about 100 mg/kg/day. A therapeutically effective amount of the active substance can be administered by an appropriate route in a single dose or multiple doses. Further, the dosages of the active substance can be proportionally increased or decreased as indicated by the exigencies of the therapeutic or prophylactic situation.

“Pharmaceutically acceptable carrier” includes any and all solvents, dispersion media, coatings, antibacterial and antifungal agents, isotonic and absorption delaying agents, and the like which are compatible with the activity of the compound and are physiologically acceptable to the subject. An example of a pharmaceutically acceptable carrier is buffered normal saline (0.15M NaCl). The use of such media and agents for pharmaceutically active substances is well known in the art. Except insofar as any conventional media or agent is incompatible with the therapeutic compound, use thereof in the compositions suitable far pharmaceutical administration is contemplated. Supplementary active compounds can also be incorporated into the compositions.

“Additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “Additional ingredients” which may be included in the pharmaceutical compositions of the invention are known in the art and described, e.g., in Remington's Pharmaceutical Sciences.

### Compounds of the Invention

Some of the compounds described herein contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well, as, their racemic and optically pure forms. Optically active (R)- and (S)-, or (D)- and (L)-isomers may be prepared using, chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

The configuration of any carbon-carbon double bond appearing herein is selected for convenience only and is not intended to designate a particular configuration; thus a carbon-carbon double bond depicted arbitrarily herein as trans may be cis, trans, or a mixture of the two in any proportion.

Disclosed herein are sulfur- and selenium-containing compounds of general Formula I

R¹-X-R²

wherein X is SO, SO₂, SeO or SeO₂, R₁ and R₂ are substituted or unsubstituted alkyl, cycloalkyl, oxaalkyl, oxoalkyl, carboxy, alkenyl, thiaalkenyl, dithiaalkenyl, substituted or unsubstituted aryl, and wherein R₁ and R₂ are the same or different or R₁ and R₂ together form a 4-6-membered heterocyclic ring structure that may be substituted or unsubstituted and polymeric forms thereof useful as anti-angiogenic agents. The sulfur and selenium compounds of the invention therefore can be used to inhibit angiogenesis to treat disorders associated with such undesired angiogenesis. Examples of sulfur and selenium containing of the invention include, but are not limited to thiete S,S-dioxide, thiolane S,S-dioxide, di-n-butyl sulfone, 2,5-dihydrothiophene S,S-dioxide, 2,4-dithiapentane 2,2-dioxide, methyl trichloromethyl sulfone, 2,4-dithiapentane 2,2,4,4-tetraoxide, 2,6-dithiaspiro[3.3]heptane 2,2,6,6-tetraoxide, selenodiacetic acid, (S)-(Z)-ajoene, (R)-(Z)-ajoene, (S)-(E)-ajoene, (R)-(E)-ajoene, dimethyl sulfone, diethyl sulfone, diphenyl sulfone, ethyl methyl sulfone, methyl phenyl sulfone, ethyl phenyl sulfone, dimethyl sulfoxide, diethyl sulfoxide, diphenyl sulfoxide, ethylmethyl sulfoxide, methyl phenyl sulfoxide, ethyl phenyl sulfoxide, dimethyl selenone, diethyl selenone, diphenyl selenone, ethyl methyl selenone, methyl, phenyl, selenone, ethyl, phenyl selenone, dimethyl selenoxide, diethyl selenoxide, diphenyl selenoxide, ethyl methyl selenoxide, methyl phenyl selenoxide, ethyl phenyl selenoxide, 3-(4-(methylsulfonyl)phenyl)propanoic acid, 3-(4-(methylsulfinyl)phenyl)propanoic acid, 3-(4-(methylselenonyl)phenyl)propanoic acid, 3-(4-(methylseleninyl)phenyl)propanoic acid; some of these examples are shown in Table 1 below.

TABLE 1

| Example | Structure |
|---|---|
| 1 | 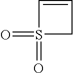 |
| 2 | 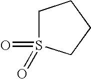 |
| 3 | 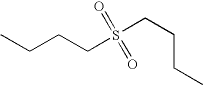 |
| 4 | 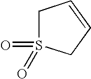 |
| 5 | 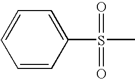 |
| 6 | 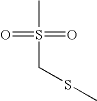 |
| 7 | 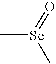 |
| 8 | 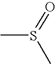 |
| 9 | 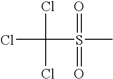 |
| 10 | 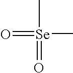 |
| 11 | 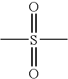 |
| 12 | 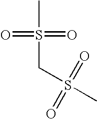 |
| 13 | 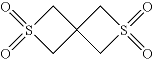 |
| 14 | 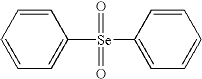 |
| 15 | 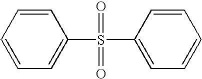 |
| 16 | 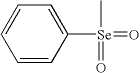 |
| 17 | 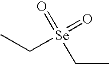 |
| 18 | 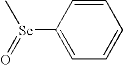 |
| 19 | 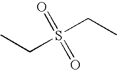 |
| 20 | 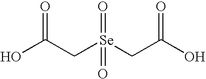 |
| 21 | 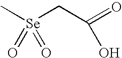 |
| 22 | 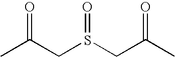 |
| 23 | 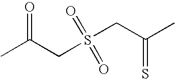 |
| 24 | 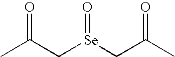 |
| 25 | 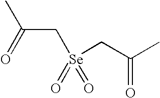 |
| 26 | 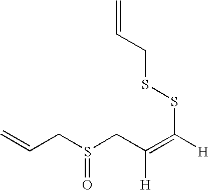 |
| 27 | 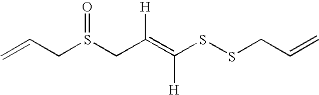 |
| 28 | 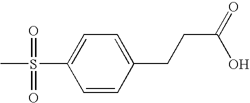 |
| 29 | 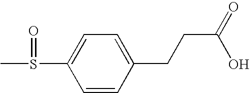 |
| 30 | 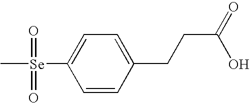 |
| 31 | 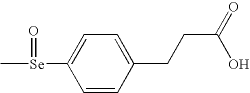 |

Compounds not intended to be encompasses by the present invention are sulindac sulfone or its sulfoxide derivative.

### Polymer Conjugates

Polymer conjugations are used to improve drug viability. While old and new therapeutics are well-tolerated, many compounds need advanced drug discovery technologies to decrease toxicity, increase circulatory time, or modify bio-distribution. One strategy for improving drug viability is the utilization of water-soluble polymers. Various water-soluble polymers have been shown to modify bio-distribution, improve the mode of cellular uptake, change the permeability through physiological barriers, and modify the rate of clearance through the body. To achieve either a targeting or sustained-release effect, water-soluble polymers have been synthesized that contain drug moieties as terminal groups, as part of the backbone, or as pendent groups on the polymer chain.

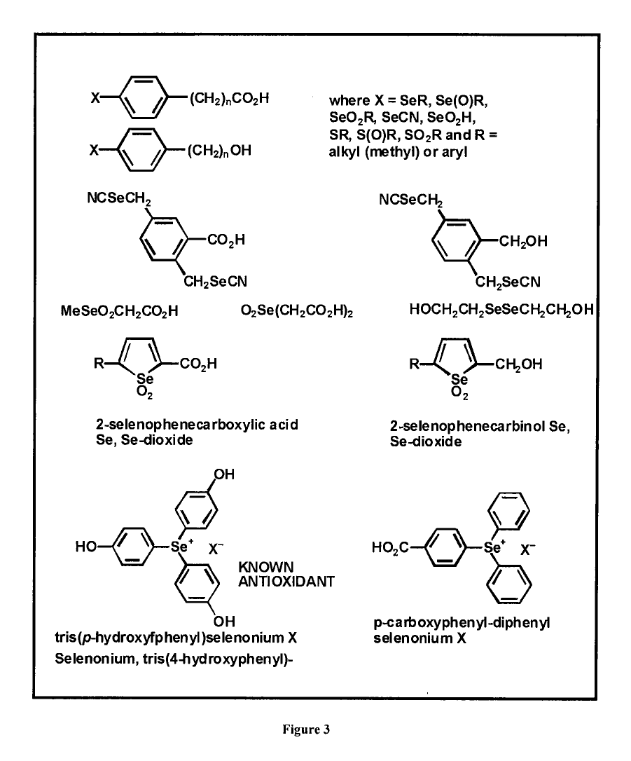

Accordingly, representative compositions of the present invention include polymeric forms of the sulfur- and selenium-containing compounds described herein. Conjugation with polymers Can be either through covalent or non-covalent linkages. In preferred embodiments; the polymer conjugation can occur through an ester linkage or an anhydride linkage. An example of a polymer conjugation through an ester linkage using polyvinyl alcohol is shown with examples selected from FIG. 3. In this preparation commercially available polyvinyl alcohol (or related co-polymers) can be esterified by treatment with the acid chloride of sulfur and selenium containing organic molecules. The hydrochloride salt is neutralized by the addition of triethylamine to afford triethylamine hydrochloride which can be washed away with water upon precipitation of the sulfur- or selenium-containing compounds ester polymer form for different analogs. The ester linkage to the polymer may undergo hydrolysis in vivo to release the active pro-angiogenesis sulfur and selenium containing organic molecules. An example of a polymer conjugation through an anhydride linkage using acrylic acid ethylene co-polymer is shown with examples selected from FIG. 3. This is similar to the previous, polymer covalent conjugation, however, this time it is through an anhydride linkage that is derived from reaction of an acrylic acid co-polymer. This anhydride linkage is also susceptible to hydrolysis in vivo to release sulfur- or selenium-containing compounds. Neutralization of the hydrochloric acid is accomplished by treatment with triethylamine and subsequent washing of the precipitated polyanhydride polymer with water removes the triethylamine hydrochloride byproduct. This reaction will lead to the formation of sulfur- or selenium-containing compounds acrylic acid co-polymer+triethylamine. Upon in vivo hydrolysis, the sulfur- or selenium-containing compounds will be released over time that can be controlled plus acrylic acid ethylene co-polymer.

Another representative polymer conjugation includes sulfur- or selenium-containing compounds conjugated to polyethylene glycol (PEG). Attachment of PEG to various drugs, proteins and Liposome has been shown to improve residence time and decrease toxicity. PEG can be coupled to active agents through the hydroxyl groups at the ends of the chains and via other chemical methods. PEG itself, however, is limited to two active agents per molecule. In a different approach, copolymers of PEG and amino acids were explored as novel biomaterials which would retain the biocompatibility properties of PEG, but which would have the added advantage of numerous attachment points per molecule and which could be synthetically designed to suit a variety of applications.

Another representative polymer conjugation includes sulfur- or selenium-containing compounds in non-covalent conjugation with polymers. A preferred non-covalent conjugation is entrapment of sulfur- or selenium-containing compounds, thereof in a polylactic acid polymer. Polylactic acid polyester polymers (PLA) undergo hydrolysis in vivo to the lactic acid monomer and this has been exploited as a vehicle for drug delivery systems in humans. Unlike the prior two covalent methods where the sulfur- or selenium-containing compounds are linked by a chemical bond to the polymer, this would be a non-covalent method that would encapsulate the sulfur- or selenium-containing compounds into PLA polymer beads. This reaction will lead to the formation of sulfur or selenium derived organic molecules containing PLA beads in water. Filter and washing will result in the formation of sulfur- or selenium-containing compounds in PLA beads, which upon in vivo hydrolysis will lead to the generation of controlled levels of sulfur- or selenium-containing compounds plus lactic acid.

Furthermore, nanotechnology can be used for the creation of useful materials and structures sized at the nanometer scale. The main drawback with biologically active substances is fragility. Nano-scale materials can be combined with such biologically active substances to dramatically improve the durability of the substance, create localized high concentrations of the substance and reduce costs by minimizing losses. Therefore, additional polymeric conjugations include nano-particle formulations of sulfur- or selenium-containing compounds thereof. In such an embodiment; nano-polymers and nano-particles can be used as a matrix for local delivery of sulfur- or selenium-containing compounds. This will aid in time controlled delivery into the cellular and tissue target.

Examples of polymeric conjugates of the sulfur- or selenium-containing compounds of the invention are provided herein and include the following examples.

Polymer conjugation through an Ester Linkage Using Polyvinyl Alcohol: In this preparation commercially available polyvinyl alcohol (or related co-polymers) can be esterified by treatment with the acid chloride form of the sulfur- and selenium-containing compounds. The so-generated hydrochloride salt is neutralized, by the addition of triethylamine to afford triethylamine hydrochloride which can be washed away with water. The ester linkage to the polymer may undergo hydrolysis in vivo to release the active pro-angiogenesis Sulfur- and selenium-containing compound.

Polymer conjugation through an Anhydride Linkage Using Acrylic Acid: This is similar to the previous polymer covalent conjugation however this time it is through an anhydride linkage that is derived from reaction of an acrylic acid co-polymer. This anhydride linkage is also susceptible to hydrolysis in vivo to release Sulfur- and selenium-containing organic molecule. Neutralization of the hydrochloric acid is accomplished by treatment with triethylamine and subsequent washing of the precipitated polyanhydride polymer with water to remove the triethylamine hydrochloride byproduct This reaction will lead to the formation of sulfur- and selenium-containing compounds acrylic acid co-polymer+triethylamine. Upon in vivo hydrolysis, the sulfur- and selenium-containing compounds will be released overtime:

Polymer compositions of sulfur- and selenium-containing compounds—Entrapment in a Polylactic Acid Polymer Polylactic acid polyester polymers (PLA) undergo hydrolysis in vivo to the lactic acid monomer, and this has been exploited as a vehicle for drug delivery systems in humans. Unlike the prior two covalent methods where the sulfur- or selenium-containing compound is linked by a chemical bond to the polymer, this would be a non-covalent method that would encapsulate the sulfur- or selenium-containing compounds into PLA polymer beads. This reaction will lead to the formation of sulfur- and selenium-containing compounds containing. PLA beads in water. Filtration and washing will result in the formation of sulfur- and selenium-containing compounds containing PLA beads, which upon in vivo hydrolysis will lead to the generation of controlled levels of sulfur- and selenium-containing compounds plus lactic acid.

Compositions of the present invention include sulfur- and selenium-containing compounds, either alone or in covalent or non-covalent conjugation with polymers. Contemplated equivalents of the compounds described above include compounds which otherwise correspond thereto, and which have the same general properties thereof (e.g., the ability to inhibit angiogenesis), wherein one or more simple variations of substituents are made which do not adversely affect the efficacy of the compound. In general, the compounds of the present invention may be prepared by the methods known to those of skill in the art or by modifications thereof, using readily available starting materials, reagents and conventional synthesis procedures. In these reactions, it is also possible to make use of variants which are in themselves known, but are not mentioned here:

Sulfur- or selenium-containing compounds, and polymeric forms in Inhibiting Angiogenesis.

The invention also provides, in another part, compositions and methods for inhibiting angiogenesis in a subject in need thereof. Conditions amenable to treatment by inhibiting angiogenesis include, for example, primary or metastatic tumors and diabetic retinopathy. The compositions can include an effective amount of monoclonal antibody to Sulfur- or selenium-containing compounds, and polymeric forms thereof. The compositions can be in the form of a sterile, injectable, pharmaceutical formulation that includes an anti-angiogenically effective amount of an anti-angiogenic substance in a physiologically and pharmaceutically acceptable carrier, optionally with one or more excipients.

In a further aspect, the invention provides methods for treating a condition amenable to treatment by inhibiting angiogenesis by administering to a subject in need thereof an amount of an anti-angiogenesis agent effective for inhibiting angiogenesis.

### Cancer-Related New Blood Vessel Growth

Examples of conditions amenable to treatment by inhibiting angiogenesis include, but are not limited to, primary or metastatic tumors. Tumor growth and metastasis are dependent upon the development of increased vasculature. In one embodiment, therefore, the invention provides a method of treating a tumor by inhibiting angiogenesis in the tumor using the sulfur- or selenium-containing compounds of the invention, and polymeric forms thereof.

### Diabetic Retinopathy

Another example of a condition amenable to treatment by inhibiting angiogenesis is diabetic retinopathy, and related ocular conditions. In such a method, fur- or selenium-containing compounds, and polymeric forms thereof.

### Methods of Treatment

The method of the invention comprises the administration of sulfur- or selenium-containing compounds or polymeric forms thereof to a subject in need of such treatment. Routes of administration may vary. Similarly, the amount of the sulfur- or selenium-containing compounds deemed to be effective in inhibiting angiogenesis will, of course, vary with the individual being treated and is ultimately at the discretion of the physician. The factors to be considered include the age and condition, of the patient being treated, the nature of the formulation, the severity of the condition and the patient's body weight.

### Formulations

The compounds described above are preferably administered in a formulation including sulfur- or selenium-containing compounds, and polymeric forms together with an acceptable carrier for the mode of administration. Any formulation or drug delivery system containing the active ingredients, which is suitable for the intended use, as are generally known to those of skill in the art, can be used. Suitable pharmaceutically acceptable carriers for oral, rectal, topical or parenteral (including subcutaneous, intraperitoneal, intramuscular and intravenous) administration are known to those of skill in the art. The carrier must be pharmaceutically acceptable in the sense of being compatible with the other ingredients of the formulation and not deleterious to the recipient thereof.

Formulations suitable for parenteral-administration conveniently include sterile aqueous preparation of the active compound, which is preferably isotonic with the blood of the recipient. Thus, such formulations may conveniently contain distilled water, 5% dextrose in distilled water or saline. Useful formulations also include concentrated solutions or solids-containing the compound of formula (I), which upon dilution with an appropriate solvent give a solution suitable for parental administration above:

For parenteral administration, a compound can be incorporated into an inert carrier in discrete units such as capsules, cachets, tablets or lozenges, each containing a predetermined amount of the active compound; as a powder or granules; or a suspension or solution in an aqueous liquid or non-aqueous liquid, e.g., a syrup, an elixir, an emulsion or a draught. Suitable carriers may be starches or sugars and include lubricants, flavorings, binders, and other materials of the same nature.

A tablet may be made by compression or molding, optionally with one or more accessory ingredients. Compressed tablets may be prepared by compressing in a suitable machine the active compound in a free-flowing form, e.g., a powder or granules, optionally mixed with accessory ingredients, e.g., binders, lubricants, inert diluents, surface active or dispersing agents. Molded tablets may be made by molding in a suitable machine, a mixture of the powdered active compound with any suitable carrier.

A syrup or suspension may be made by adding the active compound to a concentrated, aqueous solution of a sugar, e.g., sucrose, to which may also be added any accessory ingredients. Such accessory ingredients may include flavoring, an agent to retard crystallization of the sugar or an agent to increase the solubility of any other ingredient e.g., as a polyhydric alcohol, for example, glycerol or sorbitol.

Formulations for rectal administration may be presented as a suppository with a conventional Carrier, e.g., cocoa butter or Witepsol S55 (trademark of Dynamite Nobel Chemical, Germany), for a suppository base.

Alternatively, the compound may be administered in liposome or microspheres (or microparticles). Methods for preparing liposome and microspheres for administration to a patient are well known to those of skill in the art. U.S. Pat. No. 4,789,734, the contents of which are hereby incorporated by reference, describes methods for encapsulating biological materials in liposome. Essentially, the material dissolved in an aqueous solution, the appropriate phospholipids and lipids added, along with surfactants if required, and the material dialyzed or sonicated, as necessary. A review of known methods is provided by G. Gregoriadis, Chapter 14“Liposome,” Drug Carriers in Biology and Medicine, pp. 287-341 (Academic Press, 1979).

Microspheres formed of polymers or proteins are well known to those skilled in the art; and can be tailored for passage through the gastrointestinal tract directly into the blood strewn. Alternatively, the compound can be incorporated and the microspheres or composite of microspheres, implanted for slow release over a period of time ranging from days to months. See; for example, U.S. Pat. Nos. 4,906,474, 4,925,673 and 3,625,214, and Jein, TIPS 19:155-157 (1998), the contents of which are hereby incorporated by reference.

In one embodiment, the sulfur- or selenium-containing compounds, and polymeric forms thereof, and adenosine derivatives can be formulated into a liposome or microparticle, which is suitably sized to lodge in capillary beds following intravenous administration. When the liposome or microparticle is lodged in the capillary beds surrounding ischemic tissue; the agents can be administered locally to the site at which they can be most effective. Suitable liposome for targeting ischemic tissue are generally less than about 200 nanometers and are also typically uni-lamellar vesicles, as disclosed, for example, in U.S. Pat. No. 5,593,688 to Baldeschweiler, entitled “Liposomal targeting of ischemic tissue,” the contents of which are hereby incorporated by reference.

Preferred microparticles are those prepared from biodegradable polymers, such as polyglycolide, polylactide and copolymers thereof. Those of skill in the art can readily determine an appropriate carrier system depending on various factors, including the desired rate of drug release and the desired dosage.

In one embodiment, the formulations are administered via catheter directly to the inside of blood vessels. The administration can occur, for example, through holes in the catheter. In those embodiments wherein the active compounds have a relatively long half life (on the order of 1 day to a week or more), the formulations can be included in biodegradable polymeric hydrogels, such as those disclosed in U.S. Pat. No. 5,410,016 to Hubbell et al. These polymeric hydrogels can be delivered to the inside of a tissue lumen and the active compounds released over time as the polymer degrades. If desirable, the polymeric-hydrogels can have microparticles or liposome which include the active compound dispersed therein, providing another mechanism for the controlled release of the active compounds.

The formulations may conveniently be presented in unit dosage form and may be prepared, by any of the methods well known in the art of pharmacy. All methods include the step of bringing the active compound into association with a carrier, which constitutes one or more accessory ingredients. In general, the formulations are prepared by uniformly and intimately bringing the active compound into association with a liquid carrier or a finely divided solid carrier and then, if necessary, shaping the product into desired unit dosage form.

Formulations may further include one or more optional accessory, ingredient(s) utilized in the art of pharmaceutical formulations, e.g., diluents, buffers, flavoring agents, binders, surface active agents, thickeners, lubricants, suspending agents, preservatives (including antioxidants) and the like.

The invention will be further illustrated in the following non-limiting examples.

### EXAMPLES

All reagents were chemical grade and purchased from Sigma Chemical Co. (St. Louis, Mo.) or through VWR Scientific (Bridgeport, N.J.). Cortisone acetate, bovine serum albumin (BSA), and gelatin solution (2% type B from bovine skin) were purchased from Sigma Chemical Co. (St. Louis, Mo.). M199 growth medium with Earl's salts, basic FGF, Insulin-Transferrin-Selenium-G Supplement (I-T-Se) 100×, Dulbecco's phosphate buffered salt solution (PBS) with and without Ca⁺² and Mg⁺², and 0.5 M EDTA were obtained from Gibco BRL (Grand Island, N.Y.). Human umbilical vein endothelial cells (HUVEC), endothelial cell basal medium (serum-free, EBM), endothelial growth medium (EGM) (supplemented with growth factors, fetal calf serum), and 0.025% trypsin/0.01% EDTA solution were purchased from Clonetics Inc. (San Diego, Calif.). Human prostrate (TSU-Pr) tumor cells were obtained from American Type Culture Collection (Rockville, Md.). Matrigel® matrix and human collagen type III were purchased from Becton Dickinson (Bedford; MA). HEMA-3 fixative and staining solutions were purchased from Biochemical Sciences, Inc. (Swedesboro, N.J.). Fertilized chicken eggs were purchased from Charles River Laboratories, SPAFAS Avian Products & Services (North Franklin, Conn.).

### Example 1

### Inhibition of Endothelial Cell Tube Formation

Differentiation by endothelial cells was examined using a method developed by Grant et al. (Grant et al., In Vitro Cell Dev. Biol., 27A:327-336 (1991), which is hereby incorporated by reference in its entirety). Matrigel® matrix, phenol-red free (commercially available from Becton Dickinson, Bedford, Mass.) was thawed overnight at 4° C. Using cold pipette tips, 3.0 mg/well of Matrigel® matrix was placed M a cold twenty-four-multi-well plate (Falcon). Matrigel® matrix was allowed to polymerize during incubation at 37° C. for 30 minutes.

TABLE 2

| | Length(mm) ± SEM | |
|---|---|---|
| Analog | 14.08 ± 0.32 | Man % |
| FGF2 + | 43.82 ± 3.2 | Inhibition +/− SD |
| 1 | 20.64 ± 0.68 | 77.94 ± 2.28 |
| 2 | 24.03 ± 0.85 | 66.53 ± 2.87 |
| 3 | 25.41 ± 1.1 | 61.92 ± 3.57 |
| 4 | 25.25 ± 0.66 | 62.45 ± 2.24 |
| 5 | 28.81 ± 1.0 | 50.48 ± 3.48 |
| 6 | 22.91 ± 1.70 | 70.32 ± 5.71 |
| 7 | 15.63 ± 1.47 | 94.80 ± 4.94 |
| 8 | 21.77 ± 1.05 | 71.59 ± 3.8 |
| 9 | 21.95 ± 3.1 | 73.53 ± 10.49 |
| 10 | 23.35 ± 2.1 | 68.82 ± 7.07 |
| 11 | 31.48 ± 0.71 | 41.50 ± 2.5 |
| 12 | 31.47 ± 1.5 | 41.54 ± 4.93 |
| 13 | 34.61 ± 0.24 | 30.97 ± 0.80 |
| 14 | 31.91 ± 1.8 | 40.06 ± 6.12 |
| 15 | 23.43 ± 2.5 | 65.63 ± 8.9 |
| 16 | 28.78 ± 0.88 | 46.52 ± 3.2 |
| 17 | 30.98 ± 2.43 | 38.61 ± 8.7 |
| 18 | 28.22 ± 0.92 | 48.50 ± 3.3 |
| 19 | 28.46 ± 25.7 | 47.65 ± 9.2 |
| 20 | 25.11 ± 3.1 | 62.90 ± 10.5 |

Data represent mean +/− SD, n = 3, All compounds were tested at 3 μM.

### Example 2

### Neovascularization in the Chick Chorioalloantoic Membrane (CAM) and Microscopic Analysis of CAM Sections

In vivo neovascularization was examined by the method previously described by Auerbach et al. (Auerbach et al., J. Dev. Biol., 41:391-394 (1974), which is hereby incorporated by reference in its entirety). Ten-day old embryos were purchased from Spafas, Inc. (Preston, Conn.) and were incubated at 37° C. with 55% relative humidity. In the dark with the help of a candling lamp, a small hole was punctured in the shell concealing the air sac with a hypodermic needle. A second hole was punctured, in the shell on the broadside of the egg directly over an avascular portion of the embryonic membrane, as observed during candling. A false air sac was created beneath the second hole by the application of negative pressure to the first hole, which caused the chorioallantoic membrane (CAM) to separate from the shell. A window, approximately 1.0 cm², was cut in the shell over the dropped CAM with the use of a small crafts grinding wheel (Dremel, Division of Emerson Electric Company Racine, Wis.), which allowed direct, access to the underlying CAM. Filter disks of #1 filter paper (Whatman International, United Kingdom) were soaked in 3 mg/mL cortisone acetate (Sigma, St. Louis, Mo.) in a solution of 95% ethanol and water and subsequently air dried under sterile conditions. FGF2 (Life Technologies, Gaithersburg, Md.) was used to grow vessels on the CAMs of 10 day old chick embryos. Sterile filter disks adsorbed with FGF2 dissolved in PBS at 1 μg/mL were placed on growing CAMs. Sterile filter disks adsorbed with FGF2 or sulfur- or selenium-containing compounds, and polymeric forms were dissolved in PBS at 1 μg/mL were placed on growing CAM. At 24 h, test compounds or control vehicle was added directly to CAM topically.

CAM tissue directly beneath FGF2-saturated filter disk was resected from embryos treated 48 hours prior with test compound or control. Tissues were washed three times with PBS. Sections were placed in a 35-mm petri dish (Nalge Nunc, Rochester, N.Y.) and examined under a SV6 stereomicroscope (Karl Zeiss, Thornwood, N.Y.) at 50× magnification. Digital images of CAM sections adjacent to filters were collected using a 3-CCD color video camera system (Toshiba America, New York, N.Y.) and analyzed using Image-Pro Plus software (Media Cybernetics, Silver Spring, Md.).

Statistical Analysis: Statistical analysis was performed by 1-way ANOVA comparing experimental with control samples.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

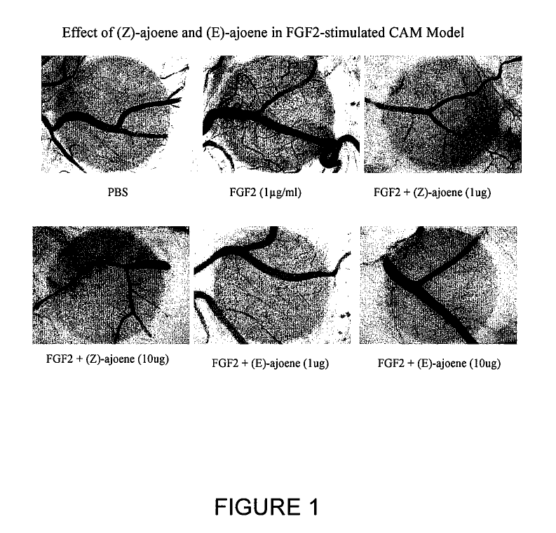

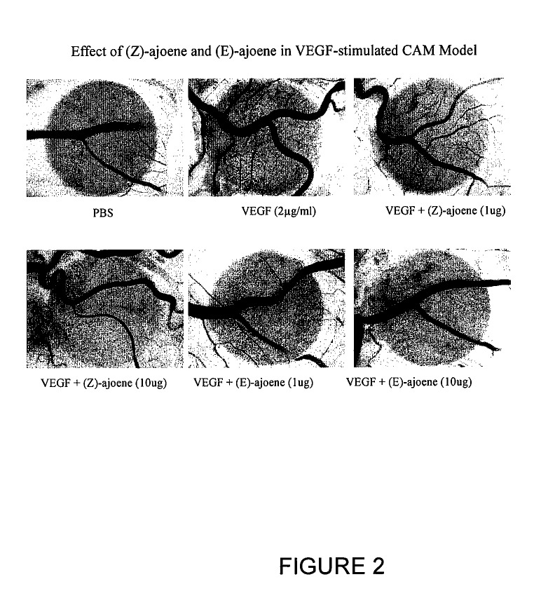

The effects of selected sulfur containing organic molecules on in vivo angiogenesis are seen in FIGS. 1 and 2.

Additionally, Tables 3 and 4 illustrates the effects of selected sulfur, and selenium compounds on angiogenesis in the CAM model.

TABLE 3

| Treatment | Branch Points ± SEM | % inhibition ± SEM |
|---|---|---|
| PBS Control | 84 ± 5.3 | |
| FGF2 (1.0 ug/ml) | 173 ± 7.5 | |
| FGF2 + # 8 | 153 ± 6.7 | 22.7 ± 7.5 |
| FGF2 + # 11 | 147 ± 9.7 | 29 ± 11 |
| FGF2 + # 1 | 149 ± 8.9 | 48.9 ± 7 |
| FGF2 + # 2 | 141.0 ± 10.69 | 55.05 ± 8.53 |
| FGF2 + # 3 | 150.88 ± 14.31 | 47.17 ± 11.42 |
| FGF2 + # 4 | 131.57 ± 11.55 | 62.58 ± 9.21 |
| PBS Control | 93.0 ± 12.13 | |
| FGF2 (1.0 ug/ml) | 180.33 ± 8.58 | |
| FGF2 + 5 | 140 ± 5.2 | 37 ± 5.9 |
| FGF2 + 6 | 158.0 ± 7.08 | 25.57 ± 8.10 |
| FGF2 + 7 | 133.5 ± 4.35 | 53.63 ± 4.98 |
| FGF2 + 20 | 148.67 ± 11.02 | 36.26 ± 12.61 |
| FGF2 + 9 | 135.83 ± 8.17 | 50.95 ± 9.36 |
| FGF2 + 10 | 143.5 ± 6.93 | 42.18 ± 7.94 |
| FGF2 + 12 | 144.83 ± 9.19 | 40.65 ± 10.53 |
| PBS Control | 93.0 ± 12.13 | |
| PBS Control | 76 ± 2.5 | |
| FGF2 (1.0 ug/ml) | 193 ± 11 | |
| FGF2 + 13 | 164 ± 5 | 24 ± 4.3 |
| FGF2 + 14 | 143.5 ± 4.7 | 41.4 ± 4.5 |
| FGF2 + 15 | 163 ± 15 | 25 ± 13 |
| FGF2 + 18 | 165 ± 5.7 | 23.8 ± 4.9 |
| FGF2 + 17 | 146 ± 8.5 | 40 ± 7 |
| FGF2 + 19 | 131 ± 17 | 53 ± 15 |

TABLE 4
Effect of Selected sulfur and selenium
Analogs in FGF2-stimulated CAM Model

| | % inhibition ± SEM |
|---|---|
| Analog | Branch point in CAM |
| FGF2 + | 10 uM |
| # 1 | 48.9 ± 7 |
| # 2 | 55.05 ± 8.53 |
| # 3 | 47.17 ± 11.42 |
| # 4 | 62.58 ± 9.21 |
| # 5 | 37 ± 5.9 |
| # 6 | 25.57 ± 8.10 |
| # 7 | 53.63 ± 4.98 |
| # 9 | 50.95 ± 9.36 |
| # 10 | 42.18 ± 7.94 |
| # 12 | 40.65 ± 10.53 |
| # 13 | 24.7 ± 4.3 |
| # 14 | 41.4 ± 4.5 |
| # 15 | 25 ± 13 |
| # 16 | 23.8 ± 4.9 |
| # 17 | 39.9 ± 7.3 |
| # 18 | 39 ± 8.3 |
| # 19 | 53 ± 15 |
| 11 | 29 ± 11 |
| 8 | 22.7 ± 7.5 |
| 20 | 36.26 ± 12.61 |

In vivo angiogenesis in Matrigel FGF₂ or Cancer cell lines implant in mice: In Vivo Murine Angiogenesis Model: The murine matrigel model are conducted according to previously described methods (Grant et al., 1991; Okada et al., 1995) and as implemented in our laboratory (Powel et al., 2000). Briefly, growth factor free matrigel (Becton Dickinson, Bedford Mass.) is thawed overnight at 4° C. and placed on ice. Aliquots of matrigel are placed into cold polypropylene tubes and FGF2, sulfur- or selenium-containing compounds, and polymeric forms thereof or cancer cells (1×10⁶ cells) will be added to the matrigel. Matrigel with Saline, FGF2, sulfur- or selenium-containing compounds, and polymeric forms thereof or cancer cells are subcutaneously injected into the ventral midline of the mice. At day 14, the mice are sacrificed and the solidified gels will be resected and analyzed for presence of new vessels. Compounds are injected subcutaneously at different doses. Control and experimental gel, implants are placed in a micro centrifuge tube containing 0.5 ml of cell lysis solution (Sigma, St Louis, Mo.) and crushed with a pestle. Subsequently, the tubes will be allowed to incubate overnight at 4° C. and centrifuged at 1,500×g for 15 minutes on the following day. A 200 μl aliquot of cell lysate are added to 1.3 ml of Drabkin's reagent solution (Sigma, St. Louis, Mo.) for each sample. The solution is analyzed on a spectrophotometer at a 540 nm. The absorption of light is proportional to the amount of hemoglobin contained in the sample.

### Example 5

Tumor growth and metastasis—Chick Chorioallantoic Membrane (CAM) model of tumor implant: The protocol is as previously described (Kim et al., 2001). Briefly, 1×10⁷ tumor cells are placed on the surface of each CAM (7 day old embryo) and incubated for one week. The resulting tumors are excised and cut into 50 mg fragments. These fragments are placed on additional 10 CAMs per group and treated topically the following day with 25 μl of compounds dissolved in PBS. Seven days later, tumors are then excised from the egg and tumor weights are determined for each CAM.

The effects of sulfur- or selenium-containing compounds, and polymeric forms thereof on tumor growth rate, tumor angiogenesis, and tumor metastasis of cancer cell, lines, can be determined.

### Example 6

Tumor growth and metastasis—Tumor Xenograft model in mice: The model is as described previously (Kerr et al., 2000; Van Waes et al., 2000; Ali et al., 2001; and Ali et al., 2001). The anti-cancer efficacy for sulfur- or selenium-containing compounds; and polymeric forms thereof at different doses and against different tumor types is then determined and compared.

Tumor growth and metastasis—Experimental Model of Metastasis: The model is as described previously (Mousa, 2002; Amirkhosravi et al., 2003a and 2003b). Briefly, B16 murine malignant melanoma cells (ATCC, Rockville; MD) and other cancer lines are cultured in RPMI 1640 (Invitrogen, Carlsbad, Calif.), supplemented with 10% fetal bovine serum, penicillin and streptomycin (Sigma, St. Louis, Mo.). Cells are cultured to 70% confluency and harvested with trypsin-EDTA (Sigma) and washed twice with phosphate buffered saline (PBS). Cells are re-suspended in PBS at a concentration of either 2.0×10⁵ cells/ml for experimental metastasis. Animals: C57/BL6 mice (Harlan, Indianapolis, Ind.) weighing 18-21 grams are used for this study. All procedures are in accordance with IACUC and institutional guidelines. The anti-cancer efficacy for sulfur- or selenium-containing compounds, and polymeric forms thereof at different doses and against different tumor types is then determined and compared.

### Example 6

Retinal Neovascularization model in mice (diabetic and non-diabetic): To assess the pharmacologic activity of a test article on retinal neovascularization, infant mice are exposed to a high oxygen environment for 7 days and allowed to recover, thereby stimulating the formation of new vessels on the retina. Test articles are evaluated to determine if retinal neovascularization is suppressed. The retinas are examined with hematoxylin-eosin staining and with at least one stain, which demonstrates neovascularization. Other stains (such as PCNA, PAS, GFAP, markers of angiogenesis, etc.) can be used. A summary of the model appears below:

### Animal Model

Infant mice (P7) and their dams are placed in a hyper-oxygenated environment (70-80%) for 7 days.
On P12, the mice are removed from the oxygenated environment and placed into a normal environment
Mice are allowed to recover for 5-7 days.
Mice are then sacrificed and the eyes collected.
Eyes are either frozen or fixed as appropriate
The eyes are stained with appropriate histochemical stains
The eyes are stained with appropriate immunohistochemical stains
Blood, serum, or other tissues can be collected
Eyes, with special reference to microvascular alterations, are examined for any and all findings. Neovascular growth will be semi quantitatively scored. Image analysis is also available.

### Example 7

A protocol disclosed in J de la Cruz et al., J Pharmacol Exp Ther 280:454-459, 1997, is used for the administration of sulfur- or selenium-containing compounds, and polymeric forms thereof, to rats that have streptozotocin (STZ)-induced experimental diabetes and diabetic retinopathy. The endpoint is the inhibition by sulfur- or selenium-containing compounds, and polymeric forms thereof, of the appearance of proliferative retinopathy.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

Suitable angiogenesis-mediated disorders in accordance with the present invention include, but are not limited to, tumors and cancer associated disorders (e.g., retinal tumor growth, benign tumors (e.g., hemangiomas, acoustic neuromas, neurofibromas, trachomas, and pyogenic-granulomas), solid tumors, blood borne tumors (e.g., leukemias, angiofibromas, and kaposi sarcoma), tumor metastases, and other cancers which require neovascularization to support tumor growth), ocular neovascular-disorders (e.g., diabetic retinopathy, macular degeneration, retinopathy of prematurity, neovascular glaucoma, corneal graft rejection, and other ocular angiogenesis-mediated disorders), inflammatory disorders (e.g., immune and non-immune inflammation, rheumatoid arthritis, chronic articular rheumatism, inflammatory bowel diseases, psoriasis, and other chronic inflammatory disorders), endometriosis, other disorders associated with inappropriate or inopportune invasion of vessels (e.g., retrolental fibroplasia, rubeosis, and capillary proliferation in atherosclerotic plaques and osteoporosis), Osler-Webber Syndrome, myocardial angiogenesis, plaque neovascularization, telangiectasia, hemophiliac joints, and wound granulation. Other diseases in which angiogenesis plays a role in the maintenance or progression of the pathological state are known to those skilled in the art and are similarly-intended to be included within the meaning of the term “angiogenesis-mediated” used herein.

In one embodiment, the sulfur- or selenium-containing compounds, and polymeric forms thereof, is used in conjunction with other angiogenesis inhibitors. Angiogenic inhibitors are known in the art and can be prepared by known methods. For example, angiogenic inhibitors include integrin inhibitory compounds such as, αν integrin inhibitory antibodies, cell adhesion proteins, or functional fragments thereof which contain a cell adhesion binding sequence. Additional angiogenic inhibitors include, for example, angiostatin (see, e.g., U.S. Pat. No. 5,639,725, which is hereby incorporated by reference in its entirety), functional fragments of angiostatin, endostatin (see, e.g., PCT publication WO 97/15666, which is hereby incorporated by reference in its entirety), fibroblast growth, factor (FGF) inhibitors, FGF receptor inhibitors, VEGF inhibitors (VEGF antibodies, VEGF trap, VEGF receptor blockers, and other mechanisms of VEGF inhibition), thrombospondin, platelet factor 4, interferon, interleukin 12, thalidomide, and compounds involved in other mechanisms for the inhibition of angiogenesis. For a description of angiogenic inhibitors, and targets set forth above, see; for example, Chen et al., Cancer Res. 55:4230-4233 (1995), Good et al., Proc. Natl. Acad. Sci. USA 87:6629-6628 (1990), O'Reilly et al., Cell 79:315-328 (1994), Parangi et al., Proc. Natl. Acad. Sci. USA 93:2002-2007 (1996), Rastinejad et al., Cell 56:345-355 (1989), Gupta et al., Proc. Natl. Acad. Sci. USA 92:7799-7803 (1995), Maione et al., Science 247:77-79 (1990), Angiolillo et al., J. Exp. Med. 182:155-162 (1995), Strieter et al., Biochem. Biophys. Res. Comm. 210:51-57 (1995); Voest et al., J. Natl. Cancer Inst. 87:581-586 (1995), Cao et al., J. Exp. Med. 182:2069-2077 (1995), Clapp et al., Endocrinology 133:1292-1299 (1993), Blood et al., Bioch. Biophys Acta. 1032:89-118 (1990), Moses et al., Science 248:1408-1410 (1990), Ingber et al., Lat Invest. 59:44-51 (1988), and U.S. Pat. Nos. 5,092,885 and 5,112,946, which are hereby, incorporated by reference in their entirety).

In another embodiment, the sulfur- or selenium-containing compounds, and polymeric forms thereof, is used in conjunction with other therapies, such as standard anti-inflammatory therapies, standard ocular therapies, standard dermal therapies, radiotherapy, tumor surgery, and conventional chemotherapy directed against solid tumors and for the control of establishment of metastases. The administration of the angiogenesis inhibitor is typically conducted during or after chemotherapy at time where the tumor tissue should respond to toxic assault by inducing angiogenesis to recover by the provision of a blood supply; and nutrients to the tumor tissue. Additionally, it is preferred to administer such angiogenesis inhibitors after surgery where solid tumors have been removed as a prophylaxis against metastasis. Cytotoxic or chemotherapeutic agents are those known in the art such as against thiotepa, alkyl sulfonate, nitrosoureas, platinum complexes, alkylators, folate analogs, purine analogs, adenosine analogs, pyrimidine analogs, substituted urea, antitumor antibiotics, microtubulle agents, and asprignase.

“Pharmaceutically acceptable salts” refers to pharmaceutically acceptable salts of sulfur- or selenium-containing compounds, and polymeric forms thereof, which salts are derived from a variety of organic and inorganic counter ions well known in the art and include, by way of example only, sodium, potassium, calcium, magnesium, ammonium, tetra-alkyl ammonium, and the like; and when the molecule contains a basic functionality, salts of organic or inorganic acids, such as hydrochloride, hydrobromide, tartrate; mesylate, acetate, maleate, oxalate and the like can be used as the pharmaceutically acceptable salt.

The compounds described above are preferably administered in a formulation including sulfur- or selenium-containing compounds, and polymeric forms thereof, together with an acceptable carrier for the mode of administration. Any formulation or drug delivery system containing the active ingredients, which is suitable for the intended use, as are generally known to those of skill in the art, can be used. Suitable pharmaceutically acceptable carriers for oral, rectal, topical or parenteral (including subcutaneous, intraperitoneal, intramuscular and intravenous) administration are known to those of skill in the art. The carrier must be pharmaceutically acceptable in the sense of being compatible with the other ingredients of the formulation and not deleterious to the recipient thereof.

Formulations suitable for parenteral administration conveniently include sterile aqueous preparation of the active compound, which is preferably isotonic with the blood of the recipient. Thus, such formulations may conveniently contain distilled water, 5% dextrose in distilled water or saline. Useful formulations also include concentrated solutions or solids containing the therapeutic of the presentation invention, which upon dilution with an appropriate solvent give a solution suitable for parental administration above.

For enteral administration, the therapeutic of the present invention can be incorporated into an inert carrier in discrete units such as capsules, cachets, tablets or lozenges, each containing a predetermined amount of the active compound; as a powder or granules; or a suspension or solution in an aqueous liquid or non-aqueous liquid, e.g., a syrup, an elixir, an emulsion or a draught. Suitable carriers may be starches or sugars and include lubricants, flavorings, binders, and other materials of the same nature.

A tablet may be made by compression or molding, optionally with one or more accessory ingredients. Compressed tablets may be prepared by compressing in a suitable machine the active compound in a free-flowing form, e.g., a powder or granules, optionally mixed with accessory ingredients, e.g., binders, lubricants, inert diluents, surface active or dispersing agents. Molded tablets may be made by molding in a suitable machine, a mixture of the powdered active compound with any suitable carrier.

A syrup or suspension may be made by adding the active compound to a concentrated, aqueous solution of a sugar, e.g., sucrose, to which may also be added any accessory ingredients. Such accessory ingredients may include flavoring, an agent to retard crystallization of the sugar or an agent to increase the solubility of any other ingredient, e.g., as a polyhydric alcohol, for example, glycerol or sorbitol.

Formulations for rectal administration may be presented as a suppository with a conventional carrier, cocoa butter or Witepsol S55 (trademark of Dynamite Nobel Chemical, Germany), for a suppository base.

Alternatively, the compound may be administered in liposome or microspheres (or microparticles). Methods for preparing liposome and microspheres for administration to a patient are well known to those of skill in the art. U.S. Pat. No. 4,789,734, which is hereby incorporated by reference in its entirety, describes methods for encapsulating biological materials in liposome. Essentially, the material is dissolved in an aqueous solution; the appropriate phospholipids and lipids added, along with surfactants if required, and the material dialyzed or sonicated, as necessary. A review of known methods is provided by G. Gregoriadis, Chapter 14, “Liposome,” Drug Carriers in Biology and Medicine, pp. 287-341 (Academic Press, 1979)₅ which is hereby incorporated by reference in its entirety.

Micro-spheres or nano-spheres formed of polymers or proteins are, well known to those skilled in the art, and can be tailored for passage through the gastrointestinal tract directly into the blood stream. Alternatively, the compound can be incorporated and the micro-spheres/nano-spheres, or composite of both, implanted for slow release over a period of time ranging from days to months. See, for example, U.S. Pat. Nos. 4,906,474, 4,925,673 and 3,625,214, and Jein, TIPS 19:155-157 (1998), which are hereby incorporated by reference in their entirety.

In one embodiment, the sulfur- or selenium-containing compounds, and polymeric forms thereof, can be formulated into a liposome or microparticle, which is suitably sized to lodge in capillary beds following intravenous administration. When the liposome or microparticle is lodged in the capillary beds surrounding ischemic tissue, the agents can be administered locally to the site at which they can be most effective. Suitable liposome for targeting ischemic tissue are generally less than about 200 nanometers and are also typically unilamellar vesicles, as disclosed, for example, in U.S. Pat. No. 5,593,688, which is hereby incorporated by reference in its entirety.

Preferred microparticles are those prepared from biodegradable polymers, such as polyglycolide, polylactide, and copolymers thereof. Those of skill in the art can readily determine an appropriate carrier system depending on various factors, including the desired rate of drug release and the desired dosage.

In one embodiment, the formulations are administered via catheter directly to the inside of blood vessels. The administration can occur, for example, through holes in the catheter'. In those embodiments wherein the active compounds have a relatively long half life (on the order of 1 day to a week or more), the formulations can be included in biodegradable polymeric hydrogels, such as those disclosed in U.S. Pat. No. 5,410,016 to Hubbell et al., which is hereby incorporated by reference in its entirety. These polymeric hydrogels can be delivered to the inside of a tissue lumen and the active compounds released over time as the polymer degrades. If desirable, the polymeric hydrogels can have microparticles or liposome which include the active compound dispersed therein, providing another mechanism for the controlled release of the active compounds.

The formulations may conveniently be presented in unit dosage form and may be prepared by any of the methods well known in the art of pharmacy. All methods include the step of bringing the active compound into association with a carrier, which constitutes one or more accessory ingredients. In general, the formulations are prepared by uniformly and intimately bringing the active compound, into association with a liquid carrier or a finely divided solid carrier and then, if necessary, shaping the product into desired unit dosage form.

In addition to the aforementioned ingredients, the formulations may further include one or more optional accessory ingredient(s) utilized in the art of pharmaceutical formulations, e.g., diluents, buffers, flavoring agents, binders, surface active agents, thickeners, honey, natural oils such as olive oil or combinations of other natural oils, lubricants, suspending agents, preservatives (including antioxidants), and the like.

We claim:

1. A method for inhibiting angiogenesis comprising contacting endothelial cells with a compound of Formula I
R₁-X-R₂
wherein
X is SO, SO₂, SeO or SeO₂;
R₁ and R₂ are substituted or unsubstituted alkyl, cycloalkyl, oxaalkyl, oxoalkyl, carboxy, alkenyl, thiaalkenyl, dithiaalkenyl, substituted or unsubstituted aryl, and wherein R₁ and R₂ are the same or different or R₁ and R₂ together form a 4-6-membered heterocyclic ring structure that may be substituted or unsubstituted.

2. The method of claim 1 wherein said compound is a sulfone of Formula II: 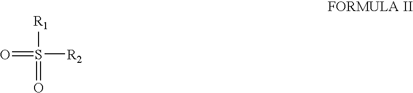
wherein
R₁ and R₂ are substituted or unsubstituted alkyl, cycloalkyl, oxaalkyl, oxoalkyl, carboxy, alkenyl, thiaalkenyl, dithiaalkenyl, substituted or unsubstituted aryl, and Wherein R₁ and R₂ are the same or different or R₁ and R₂ together form a 4-6-membered heterocyclic ring structure that may be substituted or unsubstituted.

3. The method of claim 1, wherein said compound is a sulfoxide of Formula III: 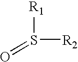
wherein
R₁ and R₂ are substituted or unsubstituted alkyl, cycloalkyl, oxaalkyl, oxoalkyl, carboxy, alkenyl, thiaalkenyl, dithiaalkenyl, substituted or unsubstituted aryl, and wherein R₁ and R₂ are the same or different or R₁ and R₂ together form a 4-6-membered heterocyclic ring structure that may be substituted or unsubstituted.

4. The method of claim 1 wherein said compound is a selenone of formula IV: 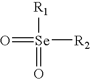
wherein
R₁ and R₂ are substituted or unsubstituted alkyl, cycloalkyl, oxaalkyl, oxoalkyl, carboxy, alkenyl, thiaalkenyl, dithiaalkenyl, substituted or unsubstituted aryl, and wherein R₁ and R₂ are the same or different or R₁ and R₂ together form a 4-6-membered heterocyclic ring structure that may be substituted or unsubstituted.

5. The method of claim 1 wherein said compound is a selenoxide of formula V: 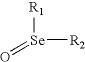
wherein.
R₁ and R₂ are substituted or unsubstituted alkyl, cycloalkyl, oxaalkyl, oxoalkyl, carboxy, alkenyl, thiaalkenyl, dithiaalkenyl, substituted or unsubstituted aryl, and wherein R₁ and R₂ are the same or different or R₁ and R₂ together form a 4-6-membered heterocyclic ring structure that may be substituted or unsubstituted.

6. The method of claim 1 wherein said compound is chosen from the group consisting of thiete S,S-dioxide, thiolane S,S-dioxide, di-n-butyl sulfone, 2,5-dihydrothiophene S,S-dioxide, 2,4-dithiapentane 2,2-dioxide, methyl trichloromethyl sulfone, 2,4-dithiapentane 2,2,4,4-tetraoxide, 2,6-dithiaspiro[3.3]heptane 2,2,6,6-tetraoxide, selenodiacetic acid, (S)-(Z)-ajoene, (R)-(Z)-ajoene, (S)-(E)-ajoene, (R)-(E)-ajoene, dimethyl sulfone, diethyl sulfone, diphenyl sulfone, ethyl methyl sulfone, methyl phenyl sulfone, ethyl phenyl sulfone, dimethyl sulfoxide, diethyl sulfoxide, diphenyl sulfoxide, ethyl methyl sulfoxide, methyl phenyl sulfoxide, ethyl phenyl sulfoxide, dimethyl, selenone, diethyl selenone, diphenyl selenone, ethyl methyl selenone, methyl phenyl selenone, ethyl phenyl selenone, dimethyl selenoxide, diethyl selenoxide, diphenyl selenoxide, ethyl methyl selenoxide, methyl phenyl selenoxide, ethyl phenyl selenoxide, 3-(4-(methylsulfonyl)phenyl)propanoic acid, 3-(4-(methylsulfinyl)phenyl)propanoic acid, 3-(4-(methylselenonyl)phenyl)propanoic acid, 3-(4-(methylseleninyl)phenyl)propanoic acid.

7. A method for treating a condition characterized by excessive or inappropriate angiogenesis comprising administering to a subject in need thereof a therapeutically effective amount of a compound of Formula I
R₁-X-R₂
wherein
X is SO, SO₂, SeO or SeO₂;
R₁ and R₂ are substituted or unsubstituted alkyl, cycloalkyl, oxaalkyl, oxoalkyl, carboxy, alkenyl, thiaalkenyl, dithiaalkenyl, substituted or unsubstituted aryl, and where R₁ and R₂ are the same or different or R₁ and R₂ together form a 4-6-membered heterocyclic ring structure that may be substituted or unsubstituted.

8. The method of claim 7 wherein said compound is a sulfone of Formula II: 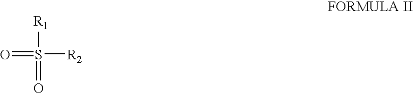
wherein
R₁ and R₂ are substituted or unsubstituted alkyl, cycloalkyl, oxaalkyl, oxoalkyl, carboxy, alkenyl, thiaalkenyl, dithiaalkenyl, substituted or unsubstituted aryl, and wherein R₁ and R₂ are the same or different or R₁ and R₂ together form a 4-6-membered heterocyclic ring structure that may be substituted or unsubstituted.

9. The method of claim 7 wherein said compound is a sulfoxide of Formula III: 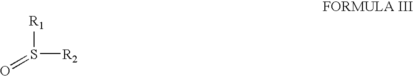
wherein
R₁ and R₂ are substituted or unsubstituted alkyl, cycloalkyl, oxaalkyl, oxoalkyl, carboxy, alkenyl, thiaalkenyl, dithiaalkenyl, substituted or unsubstituted aryl, and wherein R₁ and R₂ are the same or different or R₁ and R₂ together form a 4-6-membered heterocyclic ring structure that may be substituted or unsubstituted.

10. The method of claim 7 wherein said compound is a selenone of formula IV: 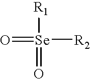
wherein
R₁ and R₂ are substituted or unsubstituted alkyl, cycloalkyl, oxaalkyl, oxoalkyl, carboxy, alkenyl, thiaalkenyl, dithiaalkenyl, substituted or unsubstituted aryl, and wherein R₁ and R₂ are the same or different or R₁ and R₂ together form a 4-6-membered heterocyclic ring structure that may be substituted or unsubstituted.

11. The method of claim 7 wherein said compound is a selenoxide of formula V: 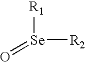
wherein
R₁ and R₂ are substituted or unsubstituted alkyl, cycloalkyl, oxaalkyl, oxoalkyl, carboxy, alkenyl, thiaalkenyl, dithiaalkenyl, substituted or unsubstituted aryl, and wherein R₁ and R₂ are the same or different or R₁ and R₂ together form a 4-6-membered heterocyclic ring structure that may be substituted or unsubstituted.

12. The method of claim 7 wherein said compound is chosen from the group consisting of thiete S,S-dioxide, thiolane S,S-dioxide, di-n-butyl sulfone, 2,5-dihydrothiophene S,S-dioxide, 2,4-dithiapentane 2,2-dioxide, methyl trichloromethyl sulfone, 2,4-dithiapentane 2,2,4,4-tetraoxide, 2,6-dithiaspiro[3.3]heptane 2,2,6,6-tetraoxide, selenodiacetic acid, (S)-(Z)-ajoene, (R)-(Z)-ajoene, (S)-(E)-ajoene, (R)-(E)-ajoene, dimethyl sulfone, diethyl sulfone, diphenyl sulfone, ethyl methyl sulfone, methyl phenyl sulfone, ethyl phenyl sulfone, dimethyl sulfoxide, diethyl sulfoxide, diphenyl sulfoxide, ethyl methyl sulfoxide; methyl phenyl sulfoxide; ethyl phenyl sulfoxide, dimethyl selenone, diethyl selenone, diphenyl selenone, ethyl methyl selenone, methyl phenyl selenone, ethyl phenyl selenone, dimethyl selenoxide, diethyl selenoxide, diphenyl selenoxide, ethyl methyl selenoxide, methyl phenyl selenoxide, ethyl phenyl selenoxide, 3-(4-(methylsulfonyl)phenyl)propanoic acid, 3-(4-(methylsulfinyl)phenyl)propanoic acid, 3-(4-(methylselenonyl)phenyl)propanoic acid, 344-(methylseleninyl)phenyl)propanoic acid.

13. The method of claim 7, wherein the condition characterized by excessive or inappropriate angiogenesis is tumor growth.

14. The method of claim 7, wherein the condition characterized by excessive or inappropriate angiogenesis is an ocular disease.

15. The method of claim 14, wherein the ocular disease is diabetic retinopathy.

16. A pharmaceutical composition for inhibiting angiogenesis comprising a therapeutically effective amount of a compound of Formula I
R₁—X-R₂
wherein
X is SO, SO₂, SeO or SeO₂;
R₁ and R₂ are substituted or unsubstituted alkyl, cycloalkyl, oxaalkyl, oxoalkyl, carboxy, alkenyl, thiaalkenyl, dithiaalkenyl, substituted or unsubstituted aryl, and wherein R₁ and R₂ are the same or different or R₁ and R₂ together form a 4-6-membered heterocyclic ring structure that may be substituted or unsubstituted and a pharmaceutically acceptable carrier.

17. The pharmaceutical composition of claim 16 comprising a therapeutically effective amount of a sulfone of Formula II: 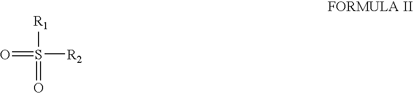
wherein
R₁ and R₂ are substituted or unsubstituted alkyl; cycloalkyl, oxaalkyl, oxoalkyl, carboxy, alkenyl, thiaalkenyl, dithiaalkenyl, substituted or unsubstituted aryl, and wherein R₁ and R₂ are the same or different or R₁ and R₂ together form a 4-6-membered heterocyclic ring structure that may be substituted or unsubstituted and a pharmaceutically acceptable carrier.

18. The pharmaceutical composition of claim 16 comprising a therapeutically effective amount of a sulfoxide of Formula III: 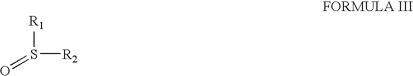
wherein
R₁ and R₂ are substituted or unsubstituted alkyl, cycloalkyl, oxaalkyl, oxoalkyl, carboxy, alkenyl, thiaalkenyl, dithiaalkenyl, substituted or unsubstituted aryl, and wherein R₁ and R₂ are the same or different or R₁ and R₂ together form a 4-6-membered heterocyclic ring structure that may be substituted or unsubstituted and a pharmaceutically acceptable carrier.

19. The pharmaceutical composition of claim 16 comprising a therapeutically effective amount of a selenone of formula IV: 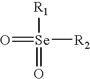
wherein
R₁ and R₂ are substituted or unsubstituted alkyl, cycloalkyl, oxaalkyl, oxoalkyl, carboxy, alkenyl, thiaalkenyl, dithiaalkenyl, substituted or unsubstituted aryl, and wherein R₁ and R₂ are the same or different or R₁ and R₂ together form a 4-6-membered heterocyclic ring structure that may be substituted or unsubstituted and a pharmaceutically acceptable carrier.

20. The pharmaceutical composition of claim 16 comprising a therapeutically effective amount of a selenoxide of formula V: 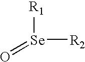
wherein
R₁ and R₂ are substituted or unsubstituted alkyl, cycloalkyl, oxaalkyl, oxoalkyl, carboxy, alkenyl, thiaalkenyl, dithiaalkenyl, substituted or unsubstituted aryl, and wherein R₁ and R₂ are the same or different or R₁ and R₂ together form a 4-6-membered heterocyclic ring structure that may be substituted or unsubstituted and a pharmaceutically acceptable carrier.

21. The pharmaceutical composition of claim 16 comprising a therapeutically effective amount of at least one compound chosen from the group consisting of thiete S,S-dioxide, thiolane S,S-dioxide, di-n-butyl sulfone, 2,5-dihydrothiophene S,S-dioxide, 2,4-dithiapentane 2,2-dioxide, methyl trichloromethyl sulfone, 2,4-dithiapentane 2,2,4,4-tetraoxide, 2,6-dithiaspiro[3.3]heptane 2,2,6,6-tetraoxide, selenodiacetic acid, (S)-(Z)-ajoene, (R)-(Z)-ajoene, (S)-(E)-ajoene, (R)-(E)-ajoene, dimethyl sulfone, diethyl sulfone, diphenyl sulfone, ethyl methyl sulfone, methyl phenyl sulfone, ethyl phenyl sulfone, dimethyl sulfoxide, diethyl sulfoxide, diphenyl sulfoxide, ethyl methyl sulfoxide, methyl phenyl sulfoxide, ethyl phenyl sulfoxide, dimethyl selenone, diethyl selenone, diphenyl selenone; ethyl methyl selenone, methyl phenyl selenone, ethyl phenyl selenone, dimethyl selenoxide, diethyl selenoxide, diphenyl selenoxide, ethyl methyl selenoxide, methyl phenyl selenoxide, ethyl phenyl selenoxide, 3-(4-(methylsulfonyl)phenyl)propanoic acid, 3-(4-(methylsulfinyl)phenyl)propanoic acid, 3-(4-(methylselenonyl)phenyl)propanoic acid, 3-(4-(methylseleninyl)phenyl)propanoic acid.

22. The method of claim 1, wherein the compound of Formula I is conjugated to a polymer selected from the group consisting of polyvinyl alcohol, acrylic acid ethylene co-polymer polyethyleneglycol and polylactic acid.

23. The method of claim 1, wherein the polymer is polyglycolide, polylactide, or co-polymers thereof.

24. The method of claim 1, wherein the compound of Formula I is encapsulated or incorporated in a microparticle, liposome or polymer.
